(19) United States
(12) Patent Application Publication
(10) Pub. No.: US 2015/0313600 A1
(43) Pub. Date: Nov. 5, 2015

(54) NERVE ABLATION DEVICE AND METHODS

(71) Applicant: Warsaw Orthopedic, Inc., Warsaw, IN (US)

(72) Inventor: William F. McKay, Memphis, TN (US)

(73) Assignee: Warsaw Orthopedic, Inc., Warsaw, IN (US)

(21) Appl. No.: 14/265,755

(22) Filed: Apr. 30, 2014

(51) Int. Cl. A61B 17/122

(52) U.S. Cl. CPC A61B 17/122

(57) ABSTRACT

A surgical implant for blocking neural regrowth following a surgical procedure is provided. The implant comprises a press, such as, for example, a clip, having a first portion and a second portion. The second portion is rotatable with respect to the first portion. The first and second portions each having an inner surface configured for engagement with tissue, such as, for example, a denatured nerve. A method of use utilizing the surgical implant is also provided.

### FIELD

Systems for treating a denatured nerve to reduce pain due to neuritis are provided. More particularly, treatment systems comprising a press for applying a compressive force to a nerve to physically block axon regrowth are provided.

### BACKGROUND

Acute and chronic pain management has been a concern for as long as medicine has been practiced. Several methods of inducing analgesia and anesthesia have been developed. For example, the use of chemical substances is perhaps the most common approach to pain relief which requires suitable substances that are effective, safe to humans, and do not cause complications or abnormal reactions. Despite advances that have been made in the field of anesthesiology, and in the field of pain relief in general, there are still some drawbacks to chemical-based approaches. For instance, the anesthetics generally available today must be administered in carefully graduated doses to assure the patient's well-being, require extended periods of fasting prior to treatment, and are often accompanied by undesirable after effects such as nausea.

One alternative to anesthetics that is commonly used for providing pain relief is ablation in which nerves and/or tissue is removed and/or destroyed. Ablation procedures can use either cold or hot ablation procedures and techniques. Various categories of ablation include but are not limited to electrical, radiation, light, radiofrequency, ultrasound, cryotherapy, thermal, microwave and hydromechanical. One form of hot ablation is radiofrequency ablation. During radiofrequency (RF) ablation, current passing through tissue from the active electrode leads to ion agitation, which is converted by means of friction into heat. The process of cellular heating includes almost immediate and irreparable cellular damage, which leads to coagulation necrosis. Because ion agitation, and thus tissue heating, is greatest in areas of highest current density (e.g., closest to the active electrode tip), necrosis is limited to a relatively small volume of tissue surrounding the RF electrode.

Another form of ablation uses cold ablation and is sometimes called cryoablation. During cryoablation, tissue is frozen or rapid freeze/thaw cycles are inflicted upon the tissue. Cryoablation allows treatment mapping pre and post procedure where areas of tissue can be mapped by limited, reversible and/or freezing. Cryoablation can be monitored and visualized on ultrasonography, CT and MRI.

Currently available nerve ablation devices and techniques leave nerve sheaths intact allowing for nerve axons to regrow across damaged tissue and restore sensory and motor function. The speed at which this regeneration occurs depends upon the method and duration of the procedure used. This restoration of sensory and motor function can occur as quickly as a few weeks to a few months. Therefore, the nerve ablation procedure needs to be repeated over and over to reduce pain symptoms that re-occur.

The use of nerve ablation devices may result in neuritis, inflammation of nerve tissue, or creation of nerve clusters, such as, for example, nerve balls. Such pain may recur in patients as many times as the nerve ablation procedure is repeated. Once a nerve is damaged or denatured, affected nerve tissue locally releases various cytokines such as nerve growth factor (NGF). NGF is essential in promoting the growth and survival of the nervous system. Once NGF is released, nerve regrowth begins in the form of damaged tissue being repaired and new nerve fibers and branches being formed. If left unchecked, the newly grown nerve tissue may grow back into a problematic portion of tissue in a patient or into a nerve ball.

Accordingly, there is a need for systems to physically block axon regrowth by positioning a device at predetermined locations along a denatured nerve to apply compressive force to the nerve. Application of such a compressive force prevents the transmission of NGF to damaged or denatured nerve tissue, and thus, prevents regrowth of nerve tissue. Further, there is a need for devices and methods that provide mechanical blocking capabilities of nerve and/or soft tissue. Devices and methods that assist in the control of neuritis in a tissue being treated are also needed.

### SUMMARY

In one embodiment, a surgical implant for blocking neural regrowth following a surgical procedure is provided. The implant comprises a press having a first portion and a second portion. The second portion is movable with respect to the first portion. The first and second portions each have an inner surface configured to engage tissue, such as, for example, a denatured nerve.

In one embodiment, a method for surgically treating pain in a patient is provided. The method comprises the step of ablating a nerve at a surgical site with an ablation probe so as to denature at least a portion of the nerve. A clip is then implanted onto the denatured portion of the nerve in order to apply a compressive force sufficient to prevent nerve growth while leaving a nerve sheath of the nerve intact.

In one embodiment, a surgical implant for blocking neural regrowth following a surgical procedure is provided. The surgical implant comprises a press, such as, for example a clip or clip, having a first portion and a second portion. The first portion is coupled to the second portion by a hinge such that the second portion is moveable relative to the first portion. The first and second portions each have an inner surface configured to engage a nerve. In some embodiments, at least a portion of the clip is coated with a non-conductive polymer. In some embodiments, a surgical tool is configured to move the clip between a first orientation in which the first portion is separated from the second portion by a first distance and a second orientation in which the first portion is separated from the second portion by a second distance, the second distance being less than the first distance. In some embodiments, the first portion comprises a locking mechanism configured to maintain the clip in the second orientation.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a side view of a clip according to an aspect of the present application;

FIG. 2 illustrates a perspective view of a clip according to one aspect of the present application;

FIG. 3 illustrates a perspective view of a clip according to one aspect of the present application;

FIG. 4A illustrates a perspective view of a clip according to one aspect of the present application;

FIG. 4B illustrates a side view the clip shown in FIG. 4A;

FIG. 5A illustrates a perspective view of a clip according to one aspect of the present application;

FIG. 5B illustrates a side view of the clip shown in FIG. 5A;

FIG. 5C illustrates a side view of a plurality of clips as shown in FIG. 5A;

FIG. 6A illustrates a perspective view of a clip according to one aspect of the present application;

FIG. 6B illustrates a perspective view of a clip according to one aspect of the present application;

FIG. 7 illustrates a cross-sectional plan view of a clip according to one aspect of the present application; and

FIG. 8 illustrates a cross-sectional plan view of the clip application shown in FIG. 7.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

Devices for preventing the regrowth of nerves for the treatment of pain are disclosed. Specifically, the disclosed devices include a mechanical device configured to place a compressive force on a denatured nerve to prevent nerve growth. The following description is presented to enable any person skilled in the art to make and use the present disclosure. Descriptions of specific embodiments and applications are provided only as examples and various modifications will be readily apparent to those skilled in the art.

In one embodiment, a novel device and methods of use for blocking regrowth of nerve tissue comprising a press, such as, for example, a clamp or clip, is provided. The described device and method of using the device eliminates the need for repeat nerve ablations on the same nerve by physically blocking axon regrowth. The clip is a ligation or small cliping device that applies a compressive force on a portion of a nerve so as to pinch the selected portion of the damaged nerve closed. Various designs of this clip are possible.

In one embodiment, a two-step procedure for using the disclosed clip is presented. First, nerve ablation is conducted using an RF, Cryo or related probe. Once the nerve is ablated, a mechanical device is applied to the denatured portion of the nerve to block axon regrowth across this section of nerve. This mechanical device is physically applied to the nerve segment so as to reduce and/or close the internal space of the nerve sheath, thereby blocking the path for the axons proximal to the ablation to reach the distal potion of the nerve. The clip also prevents a distal nerve from sending chemical signals across the blocked path to the proximal nerve so as to prevent regrowth of the nerve that could typically occur without a physical blockage. The process of destroying the nerve fibers in this area first with ablation will also eliminate any pain sensation due to the mechanical device. The other advantage of this device is that by leaving the nerve sheath intact (i.e., no disruptions in wall) nerve fibers are prevented from growing outside the blocked portion and causing painful neuritis.

In one embodiment, the clip is placed under direct visualization of the ablated nerve using a minimally invasive cannula and/or endoscope. Alternatively, the clip is placed using a percutaneous procedure by sliding an instrument within or over the introducer needle that was used to perform the nerve RF or Cryoablation procedure.

In various embodiments, the clip can be a plastic band, inflatable balloon, metal clip or clip, or shape memory clip. In other embodiments, the clip is spring loaded or mechanically deformed once in place over the nerve. In one embodiment, the clip is tied in place onto the nerve through a tightening of a band. Additionally, the clip may be held in place through the injection of a settable material around or inside the nerve.

The present disclosure may be understood more readily by reference to the following detailed description of the disclosure presented in connection with the accompanying drawings, which together form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

### DEFINITIONS

As used in the specification and including the appended claims, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes from the one particular value and/or to the other particular value.

Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure.

For purposes of the description contained herein, with respect to components and movement of components described herein, “forward” or “distal” (and forms thereof) means forward, toward or in the direction of the forward, distal end of the probe portion of the device that is described herein, and “rearward” or “proximal” (and forms thereof) means rearward or away from the direction of the forward, distal end of the probe portion of the device that is described herein. However, it should be understood that these uses of these terms are for purposes of reference and orientation with respect to the description and drawings herein, and are not intended to limit the scope of the claims.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc. and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features.

The headings below are not meant to limit the disclosure in any way; embodiments under any one heading may be used in conjunction with embodiments under any other heading.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The components of nerve blockage system 10 can be fabricated from biologically acceptable materials suitable for medical applications, including metals, synthetic polymers, ceramics and bone material and/or their composites. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of implant system 10 may be monolithically formed, integrally connected or include fastening elements and/or instruments, as described herein.

### Ligation Device

The present disclosure includes a ligation device, such as, for example, a press or a clip, that is capable of physically blocking the growth of a damaged or denatured nerve. The use of a press or clip as in the present disclosure allows for the stunting of nerve growth to treat chronic pain.

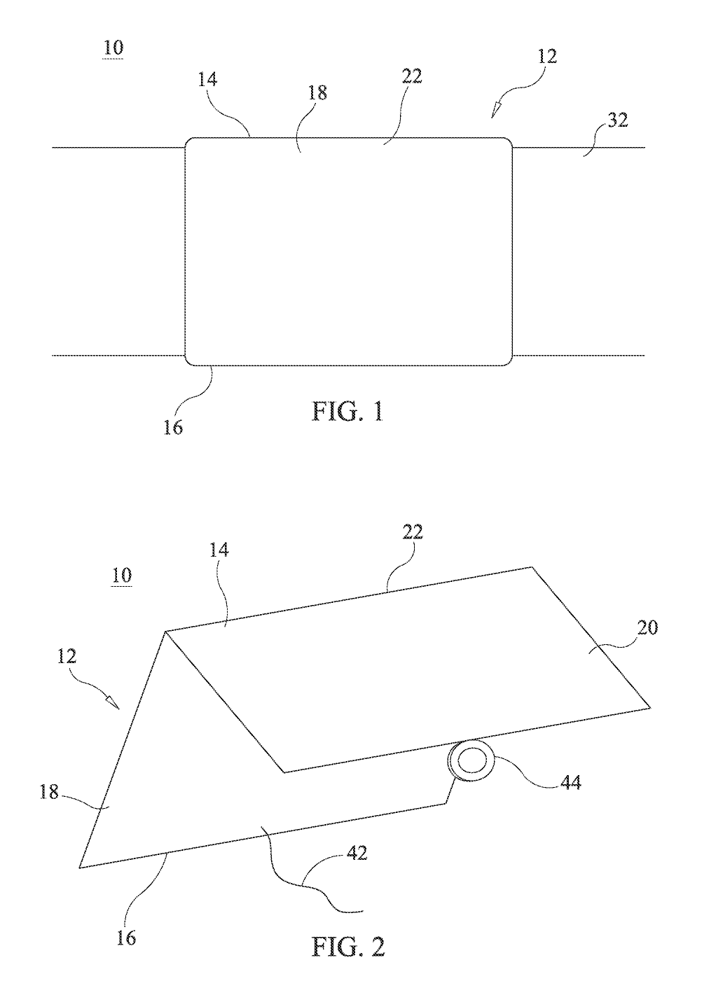

As illustrated in FIG. 1, system 10 includes a press, such as, for example, clip 12 configured to close over a damaged or denatured nerve. Clip 12 extends between a proximal end 14 and a distal end 16. In one embodiment, clip 12 comprises a foldable metal sheet configured to be folded into a first portion 18 and a second portion 20. Second portion 20 is movable with respect to first portion 18. In one embodiment, clip 12 is configured to engage a surgical tool in order to move clip 12 between a first orientation in which first portion 18 is separated from second portion 20 by a first distance and a second orientation in which first portion 18 is separated from second portion 20 by a second distance. The second distance being less than the first distance. In one embodiment, clip 12 is biased in the second orientation such that a force is required to move portions 18, 20 away from each other into the first position. Once the force is removed, portions 18, 20 are returned back to the second position thereby placing a compressive force on nerve 32 positioned there between.

In one embodiment, portions 18, 20 comprise an inner surface configured for engagement with tissue, such as, for example, nerve 32. In one embodiment, the inner surfaces of portions 18 and 20 are coated with a non-conductive material. In one embodiment, the inner surfaces of portions 18 and 20 are coated or made from a material having a high friction coefficient and are configured for engagement with tissue to enhance fixation. In some embodiments, the inner surfaces may include alternate surface configurations, such as, for example, rough, arcuate, undulating, mesh, porous, semi-porous, dimpled and/or textured according to the requirements of a particular application. In some embodiments, the inner surfaces comprise protrusions, barbs, spikes, hooks, or tips configured to engage nerve 32.

In some embodiments, clip 12 comprises a hinge 22 disposed between first portion 18 and second portion 20 that is configured to allow second portion 20 to move with respect to first portion 18. In one embodiment, hinge 22 is located toward proximal end 14. Hinge 22 provides a connection point for portions 18, 20 and an axis of movement for the movement of second portion 20 with relation to first portion 18. In one embodiment, hinge 22 comprises a continuous connection across the length of clip 12. In one embodiment, hinge 22 comprises a continuously perforated connection, such as, for example, a butt hinge or a pivot hinge.

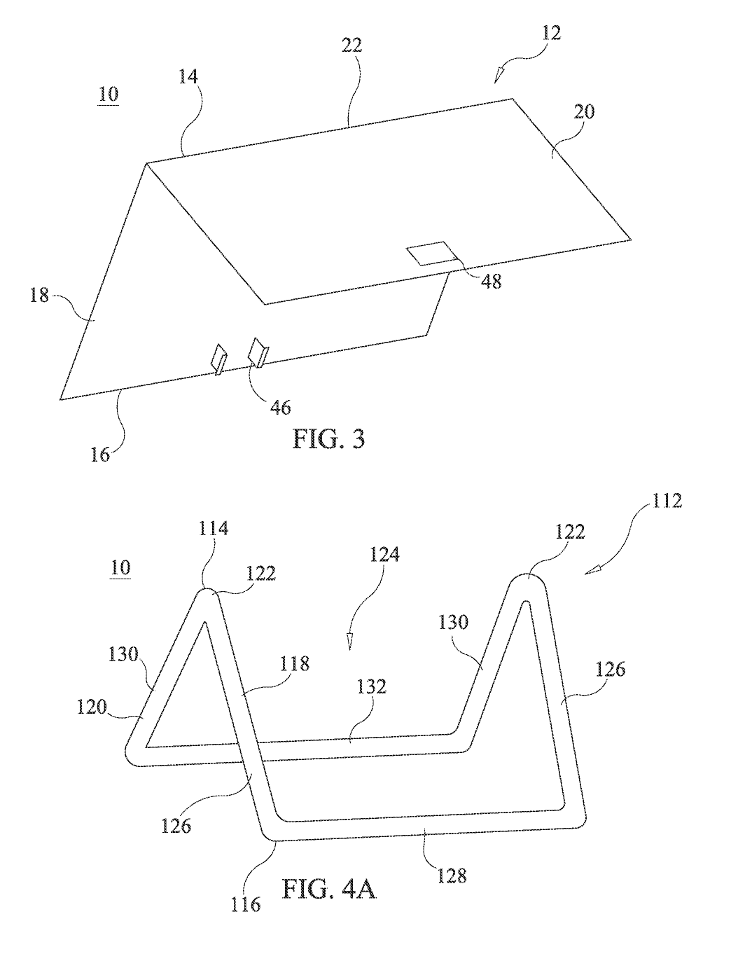

In some embodiments, clip 12 comprises a locking mechanism 40 as shown in FIGS. 2 and 3. In one embodiment, locking mechanism 40 comprises a deformable member, such as, for, example a suture 42 attached to portion 18 and a rigid loop 44 disposed on second portion 20. Suture 42 is configured to fit within ridged loop 44 so that when tied, it locks clip 12 in a closed position. Portions 18, 20 are sized such that once portions 18, 20 are moved to the second configuration over nerve 32, suture 42 and loop 44 will be disposed adjacent one another so as to facilitate the tying of suture 42 to loop 44.

In one embodiment, locking mechanism 40 comprises a protrusion 46 disposed on the inner surface of first portion 18 and an aperture defined on the second portion 20. Portions 18, 20 are sized such that once portions 18, 20 are moved to the second configuration over nerve 32, protrusion 46 and aperture 48 will be disposed adjacent one another so that protrusion 46 can be locked into aperture 48. Protrusion 46 is configured to snap into aperture 48 and lock clip 12 in the second position.

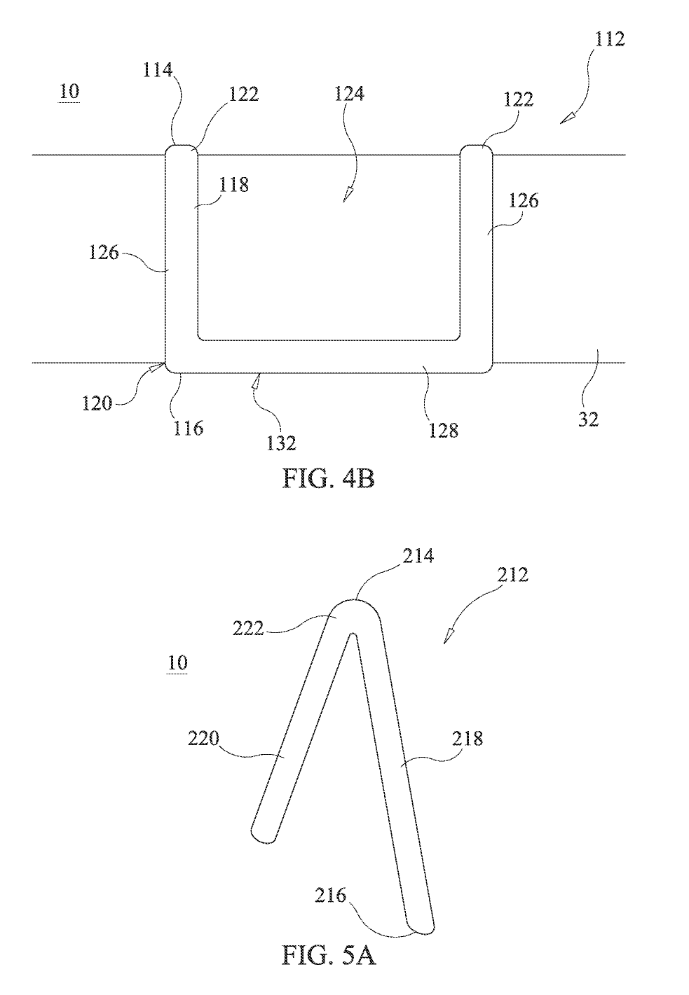

In one embodiment, as illustrated in FIGS. 4A and 4B, system 10 includes a press, such as, for example, clip 112 that is similar to clip 12. Clip 112 is configured to close over a damaged or denatured nerve and extends between a proximal end 114 and a distal end 116. In one embodiment, clip 112 comprises a foldable wire frame configured to be folded into a first portion 118 and a second portion 120. Second portion 120 is moveable with respect to first portion 118. In one embodiment, clip 112 is configured to engage a surgical tool that moves clip 112 between a first orientation in which first portion 118 is separated from second portion 120 by a first distance and a second orientation in which the first portion is separated from the second portion by a second distance. The second distance being is less than the first distance. In one embodiment, clip 112 is biased in the second orientation such that a force is required to move portions 118, 120 away from each other. Once the force is removed portions 118, 120 move to the second position thereby compressing nerve 32. In one embodiment, clip 112 is made from a deformable material that retains a closed configuration when pinched in place onto nerve 32.

Clip 112 includes a gap 124 such that portion 118 comprises two upright segments 126 configured to run transverse to nerve 32 and a horizontal segment 128 configured to provide a connection for upright segments 126. Similarly, portion 120 comprises two upright segments 130 configured to run transverse to nerve 32 and a horizontal segment 132 configured to provide a connection for upright segments 130. Gap 124 is positioned between upright segments 126, 130 such that nerve 32 is exposed through gap 124.

In one embodiment, portions 118, 120 comprise an inner surface configured to engage tissue, such as, for example, nerve 32. In other embodiments, only one of portion 118 or portion 120 comprises an inner surface configured to engage nerve 32. In one embodiment, the inner surfaces are coated with or made from a material having a high friction coefficient that enhances fixation with the nerve once the clip is in contact with nerve 32. In some embodiments, the inner surfaces may include alternate surface configurations, such as, for example, rough, arcuate, undulating, mesh, porous, semi-porous, dimpled and/or textured according to the requirements of a particular application. In various embodiments, the inner surfaces comprise protrusions, barbs, spikes, hooks, or tips configured to engage nerve 32. In one embodiment, upright segments 126, 130 comprise an inner gripping surface to grip nerve 32, and horizontal segments 128, 132 comprise a locking mechanism configured to lock clip 112 to nerve 32.

In various embodiments, clip 112 is malleable such that portions 118, 120 can be moved with respect to one another. The malleable material is configured to allow clip 112 to retain its shape, as discussed herein. Clip 112 comprises hinges 122 disposed between upright segments 126 of the first portion 118 and upright segments 130 of the second portion 120. Hinges 122 are configured to allow second portion 120 to move with respect to first portion 118. As shown in FIGS. 4A and 4B, hinges 122 are located toward proximal end 114 of clip 112. Hinge 122 provides a connection point for portions 118, 120 and connects upright segments 126 with upright segments 130. Hinges 122 also provides an axis of movement for moving second portion 120 with relation to first portion 118. In some embodiments, hinge 122 comprises a continuously perforated connection, such as, for example a butt hinge or a pivot hinge.

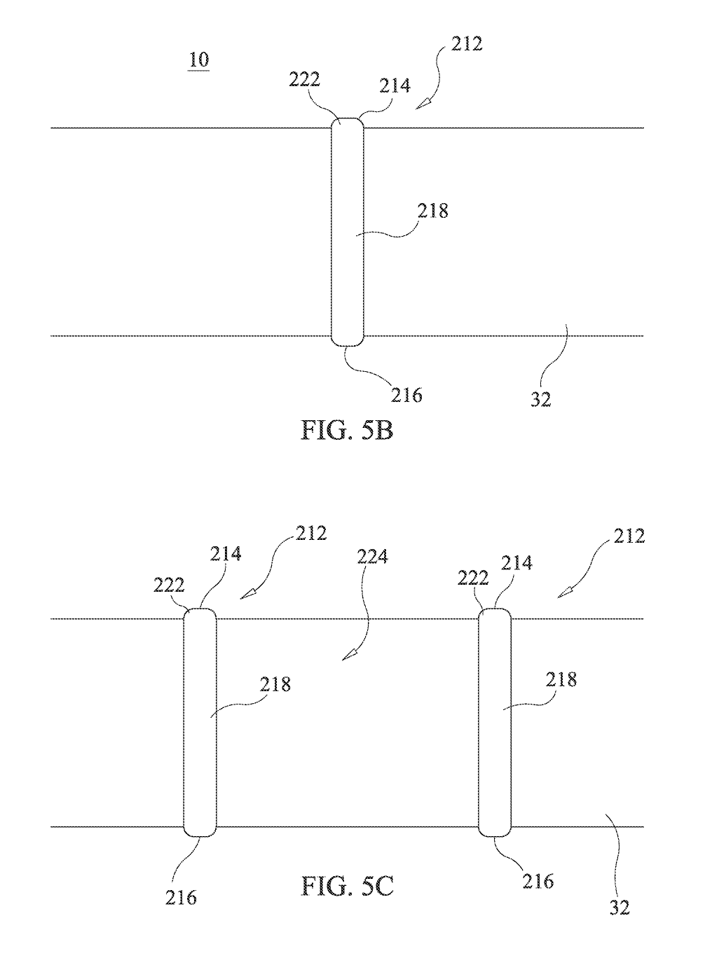

In some embodiments, as illustrated in FIGS. 5A-5C, system 10 includes a press, such as, for example, clip 212, that is similar to clips 12, 112, and is configured to close over a damaged or denatured nerve. Clip 212 extends between a proximal end 214 and a distal end 216. In one embodiment, clip 212 comprises a foldable wire frame configured to be folded into a first portion 218 and a second portion 220. Second portion 220 is movable with respect to first portion 218. In one embodiment, clip 212 is engageable with a surgical tool configured to move clip 212 between a first orientation in which first portion 218 is separated from second portion 220 by a first distance and a second orientation in which the first portion is separated from the second portion by a second distance that is less than the first distance. In one embodiment, a single clip 212 is used to place a compressive force on a portion of nerve 32.

A plurality of clips 212 can be used in series on nerve 32, as shown in FIG. 5C. Using at least two clips in series forms a space 224 between the clips 212. Space 224 can vary in length depending on the placement of the clips. In some embodiments, additional clips can be positioned relatively close to one another on nerve 32 so as to prevent nerve growth factor (NGF) from leaking from the ablated nerve and causing nerve fibers and branches being formed.

In one embodiment, portions 218, 220 comprise an inner surface configured for engagement with tissue, such as, for example, nerve 32. In other embodiments, only one of portion 218 or portion 220 comprises an inner surface configured for engagement with nerve 32. In one embodiment, the inner surfaces are coated with or made from a material having a high friction coefficient so as to enhance nerve fixation once the clip is applied. In some embodiments, the inner surfaces may include alternate surface configurations, such as, for example, rough, arcuate, undulating, mesh, porous, semi-porous, dimpled and/or textured according to the requirements of a particular application. In various embodiments, the inner surfaces comprise protrusions, barbs, spikes, hooks, or tips configured to engage nerve 32. In one embodiment, proximal end 216 of portions 218, 220 comprise a locking mechanism similar to those in FIGS. 2 and 3.

In one embodiment, clip 212 is malleable such that portions 218, 220 can be moved with respect to one another. The malleable material is configured to allow clip 212 to retain its shape, as discussed herein. In one embodiment, clip 212 comprises a hinge 222 disposed between first portion 218 and second portion 220. Hinge 222 is configured to allow second portion 220 to move with respect to first portion 218. As shown in FIG. 5A, hinge 222 is located toward proximal end 214 of clip 212. Hinge 222 provides a connection point for portions 218, 220. Hinge 222 also provides an axis or rotation for the rotation of second portion 220 with relation to first portion 218. In one embodiment, hinge 222 comprises a continuous connection across the length of clip 212. In other embodiments, hinge 222 comprises a continuously perforated connection, such as, for example, a butt hinge or a pivot hinge.

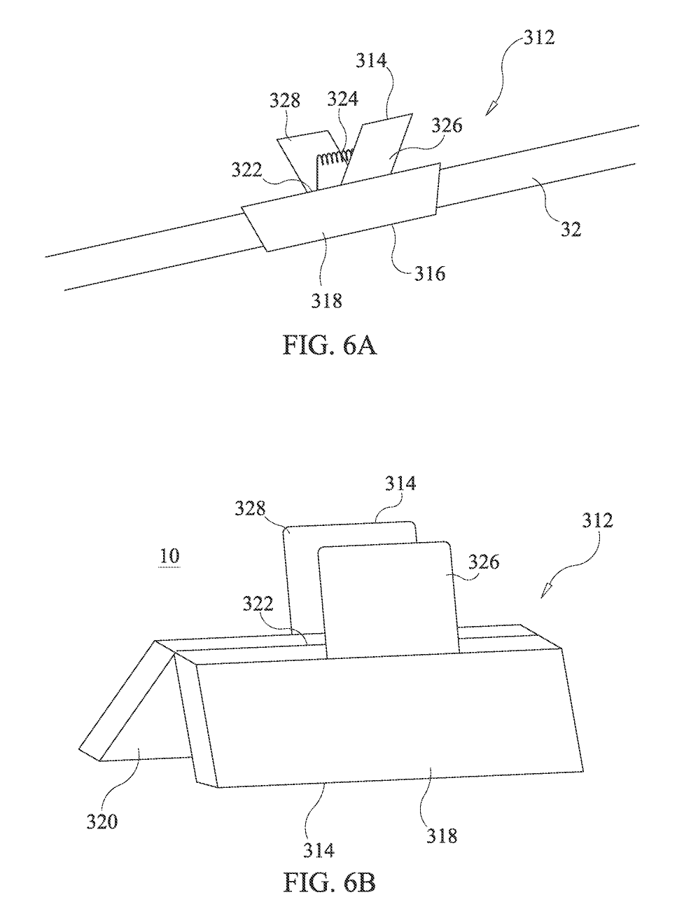

In one embodiment, as illustrated in FIGS. 6A and 6B, system 10 includes a press, such as, for example, clip 312, configured to close over a damaged or denatured portion of nerve 32. Clip 312 extends between a proximal end 314 and a distal end 316. In one embodiment, clip 312 comprises a biased clip configuration having a first portion 318 and a second portion 320. Second portion 320 is movable with respect to first portion 318. In one embodiment, clip 312 is movable between a first orientation in which first portion 318 is separated from second portion 320 by a first distance and a second orientation in which the first portion is separated from the second portion by a second distance that is less than the first distance. In one embodiment, clip 312 is biased in the second orientation such that a force is required to move portions 318, 320 away from each other. Once the force is removed, portions 318, 320 are returned back to the original and position placing a compressive force on nerve 32.

In one embodiment, clip 312 comprises a biasing member 324, such as, for example, a spring. Biasing member 324 resiliently biases clip 312 to the first orientation. In one embodiment, clip 312 comprises a tab 326 extending at an angle from first portion 318 and a tab 328 extending at an angle from second portion 320 with biasing member 324 positioned there between. Tabs 326, 328 are configured so that they can be squeezed together by a medical practitioner or a surgical tool thereby compressing biasing member 324 and moving clip 312 to the first configuration so it can be positioned over nerve 32. Once tabs 318 and 328 are released, biasing member 324 forces clip 312 into a closed, second configuration, on nerve 32 thereby applying pressure on nerve 32.

In one embodiment, portions 318, 320 comprise an inner surface configured for engagement with tissue, such as, for example, nerve 32. In other embodiments, only one of portion 318 or portion 320 comprises an inner surface configured for engagement with nerve 32. In one embodiment, the inner surfaces have a frictional surface configured to enhance fixation to nerve 32. In some embodiments, the inner surfaces may include alternate surface configurations, such as, for example, rough, arcuate, undulating, mesh, porous, semi-porous, dimpled and/or textured according to the requirements of a particular application. In various embodiments, the inner surfaces comprise protrusions, barbs, spikes, hooks, or tips configured to engage nerve 32. In one embodiment, proximal end 316 of portions 318, 320 comprise a locking mechanism.

In one embodiment, clip 312 comprises a hinge 322 disposed between first portion 318 and second portion 320 configured to allow the second portion 320 to move with respect to first portion 318. As shown in FIGS. 6A and 6B, hinge 322 is located toward proximal end 314 and between tabs 326, 328 of clip 312. Hinge 322 provides a connection point for portions 318, 320. Hinge 322 also provides an axis of movement for moving second portion 320 with relation to first portion 318. In one embodiment, hinge 322 comprises a continuous connection across the length of clip 312. In other embodiments, hinge 322 comprises a continuously perforated connection, such as, for example a butt hinge or a pivot hinge.

In some embodiments, the disclosed clips, such as, for example, clip 12, 112, 212, and/or 312, comprise a conductive material. In some embodiments, the clip comprises a non-conductive material. In some embodiments, the clip comprises a metal, such as, for example, Ti, Ti-6Al-4V, CoCr, stainless steel, or a combination thereof. In some embodiments, the clip comprises a polymer, such as, for example, PEEK, carbon-PEEK, HA-PEEK, PLA, PLDLA, or a combination thereof. It is envisioned that the design, size and shape of the clip may be adapted according to a particular application, such as, for example, spinal fixation, femur fracture stabilization, or large joint replacement fixation and/or according to the anatomic location in which the clip is to be implanted such as, for example, a pedicle, cervical spine, lumbar spine, sacroiliac joint, hip, knee, shoulder or elbow, as would be apparent to one of ordinary skill in the art.

In some embodiments, at least a portion of clips 12, 112, 212, and/or 312 is coated with a non-conductive polymer. In some embodiments, the polymer comprises one or more of polyester, polyanhydride, polyamide, polyurethane, polyurea, polyether, polysaccharides, polyamine, polyphosphate, polyphosphonate, polysulfonate, polysulfonamide, polyphosphazene, silicon oxycarbide, polysiloxane, plasma-polymerized hexamethyldisiloxane, hydrogel, polylactide, polyglycolide, or combinations thereof. In some embodiments, the polymer comprises one or more of homopolymers, copolymers or a blend of two or more homopolymers or copolymers. In some embodiments, the polymer is linear, branched, hyper-branched or dendritic.

In some embodiments, the polymer can range from a single repeat unit to about 10 million repeat units such that the polymer has a molecular weight of about 10 Daltons to about 100,000,000 Daltons. In some embodiments, the polymer comprises polymer compositions having a range or specific combination of ranges of molecular weights. In some embodiments, the polymer comprises a single polymer. In some embodiments, the polymer comprises a blend of two or more different polymers. In some embodiments, the polymer comprises linear poly(l-lactic acid) and poly(glycolic acid) having molecular weights of about 10,000 to about 1,000,000 Daltons. In some embodiments, the molecular weight of the polymer is selected to impart the polymer with a Young's modulus of about 1 GPa to about 300 GPa and/or a tensile strength of about 20 MPa to about 200 MPa.

In some embodiments, the polymer comprises a modifier or other material added to the polymer to affect the ability of the polymer to coat the press. Examples of suitable modifiers for use with the polymer include, but are not limited to resorbable fillers, antioxidants, colorants, crosslinking agents and impact strength modifiers.

In various embodiments, some or all of the clips, such as, for example, clip 12, 112, 212, and/or 312, may comprise a shape memory material. Examples of suitable shape memory materials include, but are not limited to shape memory alloys such as nickel-titanium alloys (e.g., nitinol), copper-aluminum-nickel, copper-zinc-aluminum, and iron-manganese-silicon alloys and shape memory polymers such as polyurethanes, polyurethanes with ionic or mesogenic components, block copolymers comprising polyethyleneterephthalate and polyethyleneoxide, block copolymers containing polystyrene and polybutadiene, polyesterurethanes with methylenebis and butanediol, epoxy resins.

In various embodiments, the clip may be configured in different sizes to accommodate differently sized nerves. For example, targeted nerves may have a diameter of about 1 mm to about 2 mm, about 2 mm to about 4 mm, about 1 mm to about 5 mm, or about 0.5 mm to about 5 mm and therefore different size clips can be configured to provide the proper amount of compressive force to prevent nerve growth.

In various embodiments, radiographic markers can be included on the clip to permit the user to accurately position the clip into the desired site of the patient. In this embodiment, the user may accurately position the clip in the site using any of the numerous diagnostic imaging procedures. Such diagnostic imaging procedures include, for example, X-ray imaging or fluoroscopy. Additionally, visualization of the surgical site can be enhanced through the use of a surgical cannula or an endoscope. Examples of such radiographic markers include, but are not limited to, barium, calcium phosphate, and/or metal beads. For example, the radiographic marker can be ring-shaped or dispersed as small pellets throughout the clip.

In various embodiments, the clip may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of spacer 10 relative to the absence of the material or topography.

### Methods of Blocking a Denatured or Damaged Nerve

The present disclosure also provides for methods of blocking a nerve from regrowing after it has been denatured or damaged in a surgical procedure. In one embodiment, a method for surgically treating pain in a patient is disclosed comprising the step of ablating a nerve at a surgical site with an ablation probe to denature at least a portion of a nerve. In various embodiments, the ablation probe comprises a heating probe or a cryogenic freezing probe.

The methods comprise disposing a probe at a surgical site, wherein the probe comprises an interior surface defining an internal passage and an exterior surface comprising a tip. The internal passage has a filament comprising an opening configured to heat or to cool the exterior surface of the probe to a selected temperature. The probe is positioned adjacent to a nerve and/or soft tissue at a selected location in the body of a patient so as to ablate the nerve and/or soft tissue.

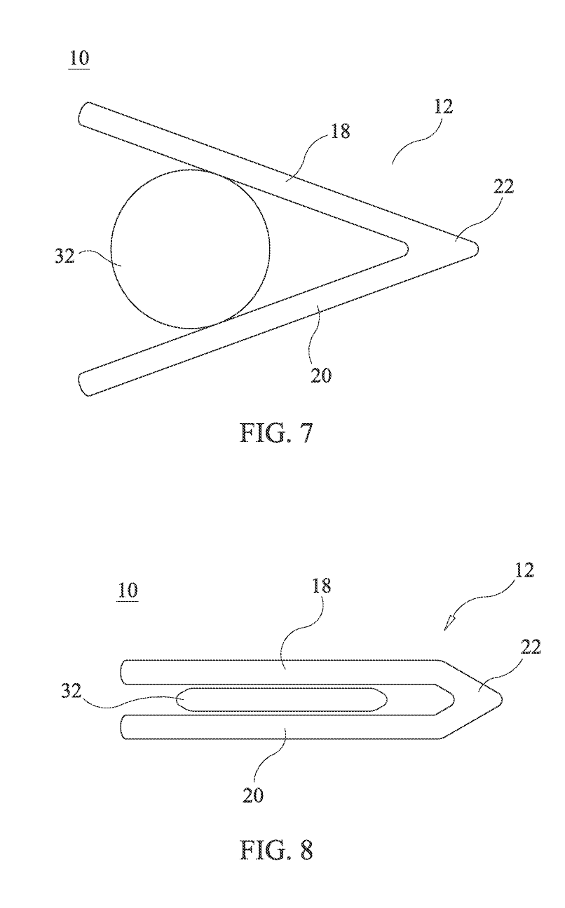

Once the nerve is ablated, the clip is placed on the denatured portion of nerve 32 to apply a compressive force sufficient to prevent nerve growth while leaving a nerve sheath of nerve 32 intact. As shown in FIGS. 7 and 8, the application of the compressive force also physically reduces and closes an internal space of the nerve sheath so as to block a path for axons proximal to the denatured portion of the nerve to reach a distal portion of nerve 32. Furthermore, the compressive force also prevents the distal portion of nerve 32 from sending chemical signals across the blocked path to a proximal portion of nerve 32 that is responsible for regrowth.

In one embodiment, the clip is implanted onto nerve 32 through the use of an implantation tool to move the clip from a first orientation in which a first potion, such as, for example, portion 18, of the clip is separated from a second portion, such as, for example, portion 20, of the clip by a first distance and a second orientation in which the first portion is separated from the second portion by a second distance that is less than the first distance. In one embodiment, the clip is configured to be gripped by a hand of a medical practitioner to move the clip between the first and second configurations. In the first orientation, the inner surfaces of the first and second portions, such as, for example portions 18, 20, of the clip are spaced apart from one another so that the clip can be positioned onto nerve 32. Once positioned on nerve 32, the clip is moved to the second orientation such that the inner surfaces of the first and second portions engage nerve 32. In one embodiment, the inner surfaces have a frictional surface configuration for engagement with tissue to enhance fixation. In some embodiments, the inner surfaces may include alternate surface configurations, such as, for example, rough, arcuate, undulating, mesh, porous, semi-porous, dimpled and/or textured according to the requirements of a particular application. In various embodiments, the inner surfaces comprise protrusions, barbs, spikes, hooks, or tips configured to engage nerve 32.

In one embodiment, the clip is resiliently biased in the second position such that once in place over nerve 32, the clip will maintain a compressive force to block regrowth of nerve 32. In one embodiment, a locking mechanism on the inner surfaces of the first and second portions is used to maintain the clip in the second orientation once the clip is positioned over nerve 32.

As discussed herein, the locking mechanism 40 comprises a deformable member, such as, for, example a suture 42 disposed with first portion 18 and a rigid loop 44 disposed to second portion 20. Suture 42 locks clip 12 in place when it is tied through loop 44. Portions 18, 20 are sized such that once portions 18, 20 are moved to the second configuration over nerve 32, suture 42 and loop 44 will be disposed adjacent one another to facilitate engaging suture 42 to loop 44. In one embodiment, a settable material can be applied around or inside the nerve to hold the clip in place and/or prevent leakage of NGF.

In one embodiment, locking mechanism 40 comprises a protrusion 46 disposed to the inner surface of first portion 18 and an aperture defined by second portion 20. Portions 18, 20 are sized such that once portions 18, 20 are moved to the second configuration over nerve 32, protrusion 46 and aperture 48 will be disposed adjacent one another to facilitate engaging protrusion 46 to aperture 48. Protrusion 46 is configured to snap into aperture 48 when portion 20 is rotated such that protrusion 46 comes into contact with aperture 48.

In some embodiments, the user may accurately position the clip in the site using any of the numerous diagnostic imaging procedures. Such diagnostic imaging procedures include, for example, X-ray imaging or fluoroscopy. Additionally, visualization of the surgical site can be enhanced through the use of a surgical cannula or an endoscope. In various embodiments, the clip may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of spacer 10 relative to the absence of the material or topography.

In various embodiments, kits are provided that include device 10. The kits can include at least one probe, at least one clip and at least one insertion instrument. In some embodiments, the kits include different sized clips for different sized nerves. In some embodiments, the probe and/or the insertion instrument in the kits is reusable for multiple procedures after cleaning and sterilization.

Specific clinical application of this instrument include destruction of nerves causing facet and discogenic back and leg pain, destruction of soft tissue causing stenosis pain symptoms, and many other orthopedic and oral maxillofacial pain. Many other painful conditions associated with arthroscopic, otolaryngological or spinal procedures that use ablation can use the devices and methods described herein.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A surgical implant for blocking neural regrowth following a surgical procedure comprising,
a clip having a first portion and a second portion, the second portion moveable with respect to the first portion, the first and second portions each having an inner surface configured for engagement with tissue.

2. A surgical implant according to claim 1, further comprising a hinge disposed between the first portion and the second portion configured to allow the second portion to move with respect to the first portion.

3. A surgical implant according to claim 1, wherein the clip is configured to engage a surgical tool to move the clip between a first orientation in which the first portion is separated from the second portion by a first distance and a second orientation in which the first portion is separated from the second portion by a second distance, wherein the second distance is less than the first distance.

4. A surgical implant according to claim 3, wherein the clip is made from a deformable material that retains the second orientation after the surgical tool disengages the clip.

5. A surgical implant according to claim 3, wherein the clip is biased to the second orientation by a biasing member.

6. A surgical implant according to claim 1, further comprising a locking mechanism configured to maintain the clip in a closed orientation to prevent neural regrowth.

7. A surgical implant according to claim 1, wherein the first and second portions of the clip comprises a deformable wire frame.

8. A surgical implant according to claim 1, wherein the first and second portions of the clip comprises a metal sheet connected together by a hinge disposed between the first portion and the second portion configured to allow the second portion to move with respect to the first portion.

9. A surgical implant according to claim 1, wherein at least a portion of the clip is coated with a non-conductive polymer.

10. A surgical implant according to claim 1, wherein at least a portion of the clip comprises a shape memory material.

11. A surgical implant according to claim 1, wherein at least a portion of the clip is coated with radiomarkers.

12. A method for surgically treating pain in a patient, the method comprising:
ablating a nerve at a surgical site with an ablation probe to denature at least a portion of the nerve; and
implanting a clip at a location on the denatured portion of the nerve to apply a compressive force sufficient to prevent nerve growth while leaving a nerve sheath of the nerve intact.

13. A method according to claim 12, further comprising implanting a second clip onto the denatured portion of the nerve at a location different than the location of the first clip so as to physically apply a compressive force to reduce and/or close an internal space of the nerve sheath so as to block a path for axons proximal to the denatured portion of the nerve to reach a distal portion of the nerve thereby blocking nerve growth.

14. A method according to claim 13, further comprising locking the first and second clips on the denatured nerve so as to prevent the distal portion of the nerve from sending chemical signals across the blocked path to a proximal portion of the nerve thereby blocking nerve growth.

15. A method according to claim 13, wherein the ablation probe is an RF heating probe.

16. A method according to claim 13, wherein the ablation probe is a cryogenic freezing probe.

17. A method according to claim 13, wherein:
implanting the clip comprises engaging an implantation tool to the clip to move the clip from a first orientation in which a first portion of the clip is separated from a second portion of the clip by a first distance to a second orientation in which the first portion is separated from the second portion by a second distance that is less than the first distance; and
positioning the clip over the nerve when the clip is in the first configuration and manipulating the implant tool so as to move the clip to the second orientation so that the inner surfaces of the first and second portions apply pressure on the nerve.

18. A method according to claim 17, further comprising activating a locking mechanism to maintain the clip on the nerve in the second orientation.

19. A method according to claim 17, further comprising positioning a second clip over the nerve and manipulating the implant tool to move the clip to the second orientation to apply pressure to the nerve.

20. A surgical implant for blocking neural regrowth following a surgical procedure, comprising:
a clip having a first portion and a second portion, the first portion being coupled to the second portion by a hinge such that the second portion is moveable relative to the first portion, the first and second portions each having an inner surface configured for engagement with a nerve, and
wherein at least the inner surface of the clip is coated in a non-conductive polymer,
wherein the clip is configured to engage a surgical tool that moves the clip between a first orientation in which the first portion is separated from the second portion by a first distance and a second orientation in which the first portion is separated from the second portion by a second distance that is less than the first distance,
wherein the first portion comprises a locking mechanism configured to engage a locking mechanism on the second portion to maintain the clip in the second orientation when positioned on the nerve.
